(19) United States
(12) Patent Application Publication
(10) Pub. No.: US 2013/0331928 A1
(43) Pub. Date: Dec. 12, 2013

(54) Dialysis Graft with Thromboses Prevention Arrangement

(71) Applicants: Chen Yang, Taoyuan County (TW); Yen-Ni Hung, Taipei City (TW); Mao-Tsun Wu, Taichung City (TW)

(72) Inventors: Chen Yang, Taoyuan County (TW); Yen-Ni Hung, Taipei City (TW); Mao-Tsun Wu, Taichung City (TW)

(21) Appl. No.: 13/965,336

(22) Filed: Aug. 13, 2013

Related U.S. Application Data
(63) Continuation-in-part of application No. 12/270,002, filed on Nov. 13, 2008.

(51) Int. Cl. A61F 2/06

(52) U.S. Cl. CPC A61F 2/064
USPC 623/1.35

(57) ABSTRACT

A dialysis graft includes an artery connection section having one end connected to an artery; and a vein connection section having both ends connected to two sections of a vein respectively. Diameter of either end of the vein connection section is equal to that of the corresponding section of the vein. The other end of the artery connection section is integrally formed with the vein connection section and communicates therewith. An enlargement for preventing stenosis and thromboses is formed at the connection of the artery connection section and the vein connection section. The dialysis graft further includes lines, ring markings, and markings for providing a visual help to a physician when endovascular therapy is needed.

### CROSS-REFERENCED TO RELATED APPLICATIONS

This application is a continuation-in-part of application Ser. No. 12/270,002 filed Nov. 13, 2008 entitled “Dialysis Graft With Thromboses Prevention Arrangement” and which is hereby incorporated herein by reference in its entirety.

### STATEMENT REGARDING FEDERALLY SPONSORED RESEARCH OR DEVELOPMENT

Not applicable

### BACKGROUND OF THE INVENTION

1. Field of Invention

The invention relates to dialysis grafts and more particularly to a dialysis graft having an enlargement at its outlet to prevent thrombi formation and facilitate the blood flow through the dialysis graft so as to reduce the cost of thrombectomy and improve the quality of life among end-stage renal disease patients.

2. Description of Related Art

In order to have a secure vascular access for hemodialysis, many patients with end stage renal disease need to receive dialysis graft implantation. According to the data provided by Taiwan Dialysis Foundation in 2005, 19,649 males and 2,256 females have to receive hemodialysis, respectively. Dialysis grafts are crucial for end stage renal disease patients. Complications of graft that may prohibit regular hemodialysis and that may be fatal to renal failure patients. Most common complications (e.g., about 80%) related to the adverse effect of traditional dialysis grafts are thromboses at venous outlet end of dialysis graft.

A conventional dialysis graft 70 is shown in FIGS. 5 and 6. The dialysis graft 70 is elongated and has one end connected to an artery 80 and the other end connected to a vein 90. However, intimal hyperplasia would be encountered and caused venous outlet stenosis at the connection of the dialysis graft 70 and the vein 90 as detailed below.

Because of the compliance difference between graft (70) and native vein (90), the possible vibration may be occurred and possible endothelial injury would be encountered. Furthermore, the endothelial injury would lead tissue in-growth that caused stenosis of venous outlet. As the result, endothelial injury would be activated coagulation pathway that induced thrombi (100) formation. The formation of thrombi would lead a vicious cycle that lead further graft venous outlet stenosis and diminish the blood flow.

A typical solution for venous outlet stenosis is to provide a flared portion at the connection site of the dialysis graft outlet that reduces the turbulent flow. However, stitching is necessary at the end of the dialysis graft and this may inevitably decrease the diameter of the outlet of the dialysis graft. As a result, the end of the dialysis graft narrow again. Moreover, the stitching is technical demand and time consuming. Thus, the need for improvement still exists.

### SUMMARY OF THE INVENTION

It is therefore one object of the invention to provide a dialysis graft comprising a vein connection section having first and second ends and including a smooth surfaces adapted to slip and connect to two sections of a vein respectively; an artery connection section extending out of an intermediate portion of the vein connection section and communicating therewith, the artery connection section and the vein connection section forming a dialysis graft of T-shape, the artery connection section having a length greater than that of the vein connection section, the artery connection section having a third end connected to the artery, and the artery connection section being inclined at an angle with respect to the vein connection section at the connection to the artery to define an acute angle side and an obtuse angle side; an enlargement for preventing thromboses formed at the obtuse angle side of the connection; wherein each of the artery and vein connection sections include marked lines on both sides on outer surface respectively, and each line has a plurality of equally spaced markings along its length; wherein each of the lines of the artery connection section and the corresponding one of the lines of the vein connection section are joined at the enlargement; wherein each of the artery and vein connection sections have a smooth inner surface; wherein the vein connection section includes two rings proximate both the first and second ends respectively; and wherein each of the lines of the vein connection section is perpendicular to each of the rings.

In addition, we utilized dye that could be visible under fluoroscopy to mark at lateral aspect of T-shape graft. The straight mark line was located at mid-portion of lateral aspect of artery and venous connection section. In addition, there were 2 complete ring marks at both ends of venous connection section.

Preferably, the dialysis graft is made by Polytetraflouroethylene (PTFE).

Preferably, a distance between the ring and the adjacent end of the vein connection section is 0.5 cm, a distance between the ring and the marking adjacent thereto is 0.5 cm, and a distance between any two adjacent markings is 0.5 cm.

Preferably, these markings are colorful that visible by naked eyes and also could be seen under fluoroscopy.

The invention has the following advantages as compared with the prior art. The T-shaped dialysis graft of the invention can prevent vibration that cause by compliance difference between graft and native vessel. As a result, the endothelial injury could be avoided and tissue in-growth could be minimized. Neither venous outlet stenosis nor turbulent blood flow would be encountered. This would prevent thrombi formation and avoid repeat thrombectomy.

The marks of the graft are not only preventing graft torsion but also quantify the length during operation. In addition, they also could be seen under fluoroscopy and lead endovascular therapy more easily to perform.

The above and other objects, features and advantages of the invention will become apparent from the following detailed description taken with the accompanying drawings.

### BRIEF DESCRIPTION OF THE DRAWINGS

FIG. 1 is a schematic perspective view of a preferred embodiment of a dialysis graft according to the invention connecting to a vein;

FIG. 1A is a detailed view of the area in circle A in FIG. 1;

FIG. 2 is a schematic perspective view of the dialysis graft interconnecting an artery and the vein with blood flow being illustrated;

FIG. 3 is an exploded view of FIG. 2;

FIG. 4 is a side elevation of the vein connection, the enlargement, and a portion of the artery connection section of FIG. 3;

FIG. 5 is a schematic perspective view of a conventional dialysis graft interconnecting an artery and a vein; and

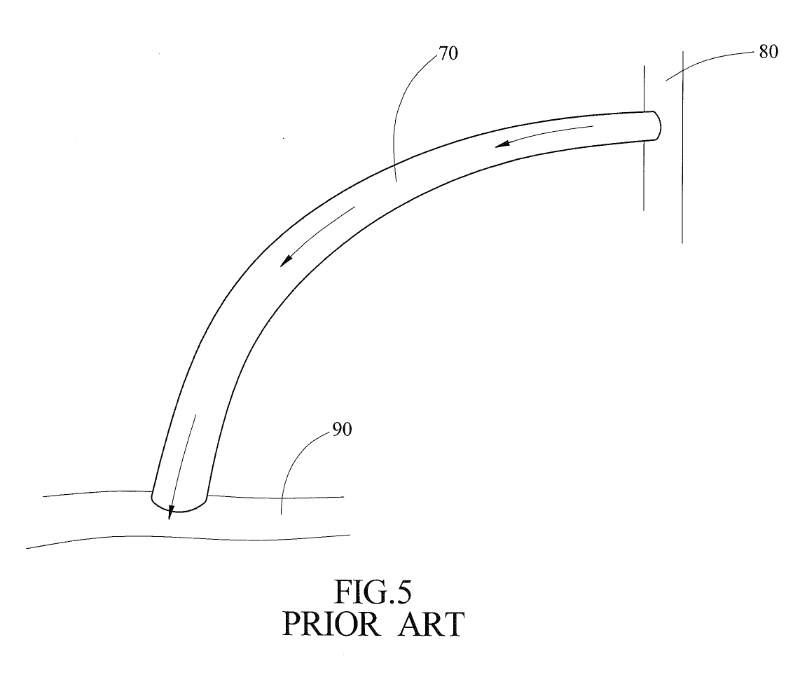

FIG. 6 is a fragmentary view in part section of FIG. 5 schematically showing the formation of thrombi at a plurality of sites at the connection of the dialysis graft and the vein.

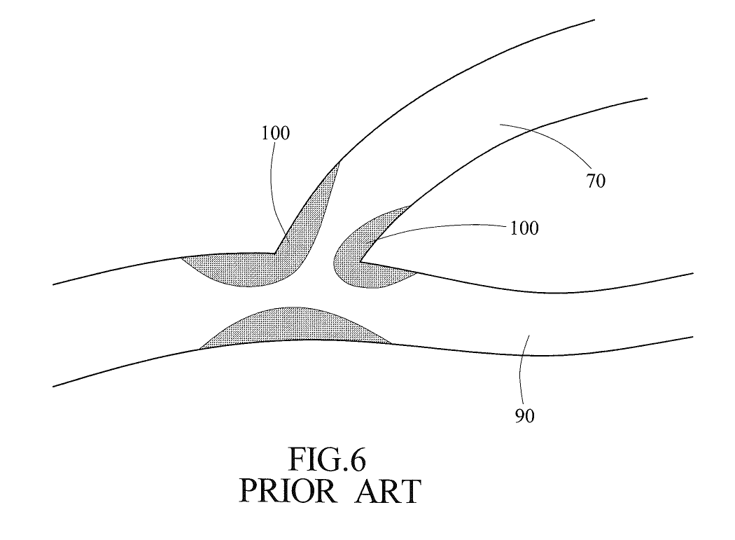

### DETAILED DESCRIPTION OF THE INVENTION

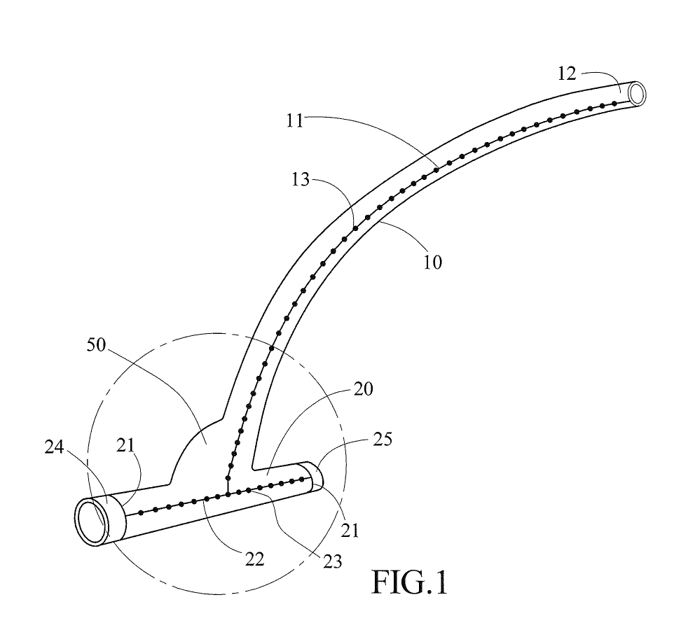

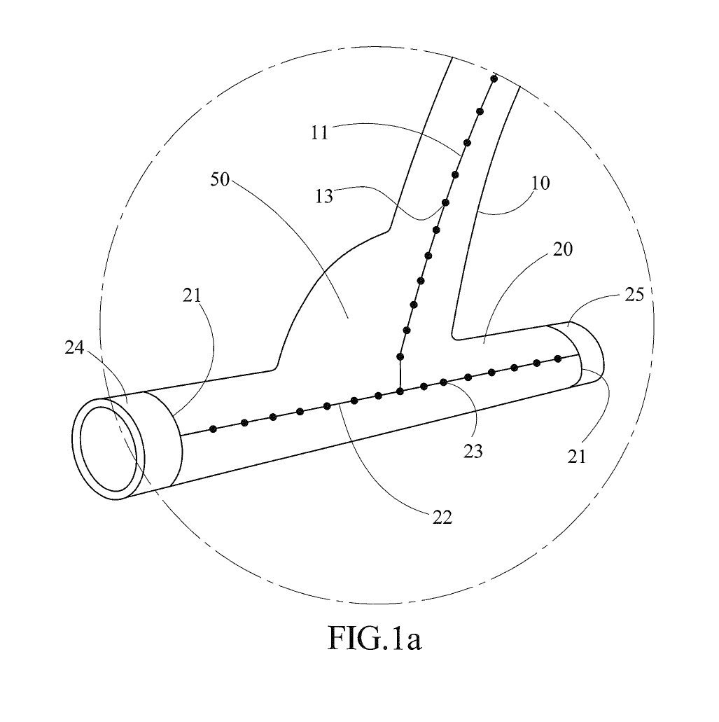

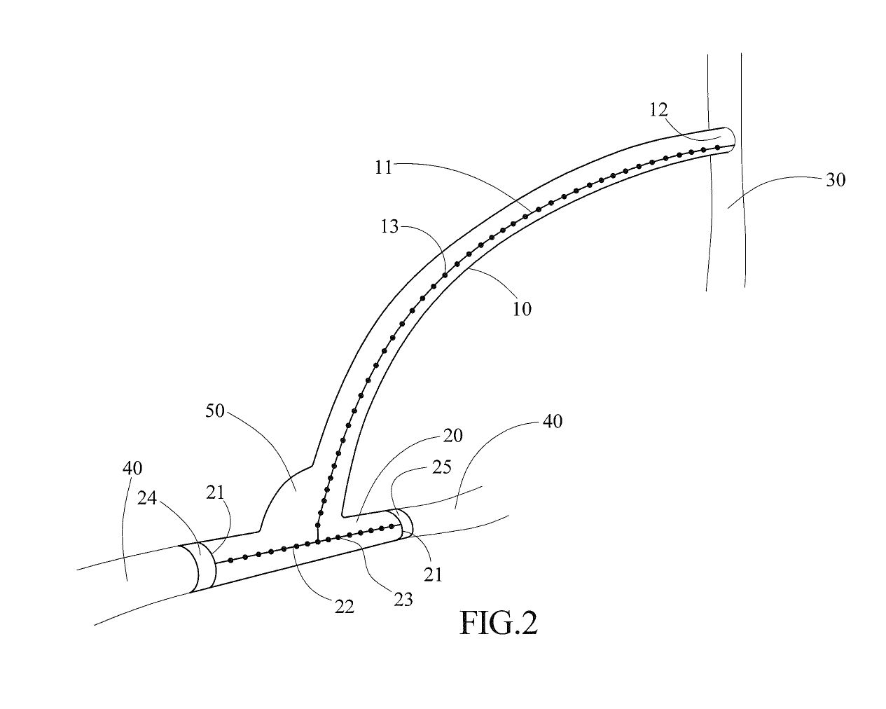

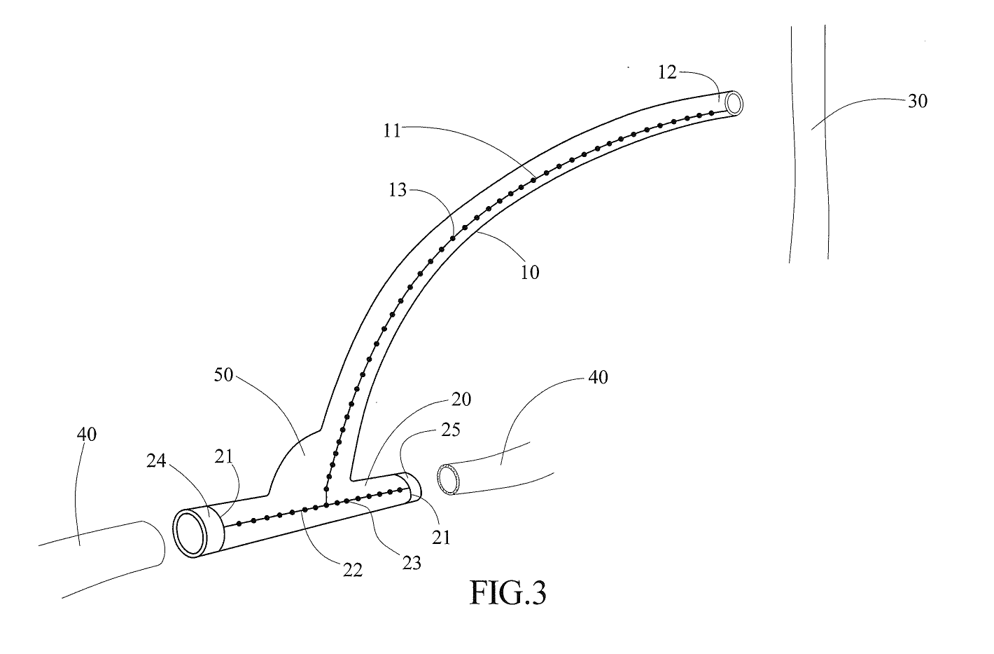

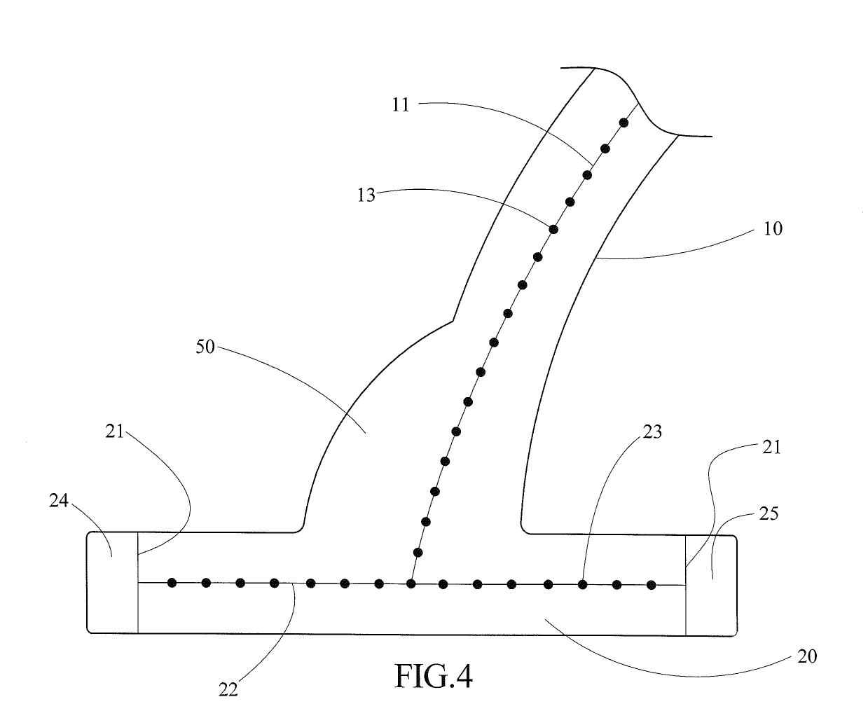

Referring to FIGS. 1 to 4, a dialysis graft in accordance with a preferred embodiment of the invention is shown. The dialysis graft comprises an artery connection section 10 and a vein connection section 20. Each of above components will now be described in detail below.

The vein connection section 20 has first and second ends 24, 25 adapted to connect to two sections of a vein 40 respectively. Diameters of the vein connection section 20 between the first and second ends 24, 25 are constant and are equal to that of the two sections of the vein 40. That is, the connection of the vein connection section 20 and either section of the vein 40 has a smooth inner surface. The other end of the artery connection section 10 is integrally formed with the vein connection section 20 and communicates therewith. An enlargement 50 is formed at the connection of the artery connection section 10 and the vein connection section 20. Further, the connection of the artery connection section 10 and the vein connection section 20 is shaped as a T. The artery connection section 10 is longer than the vein connection section 20. The artery connection section has a third end 12 connected to the artery 30. The artery connection section 10 is inclined at an angle with respect to the vein connection section 20 at the connection to the artery 30 to define an acute angle side and an obtuse angle side. The enlargement 50 is capable of preventing thrombi from being formed at the connection of the artery connection section 10 and the vein connection section 20.

Preferably, the artery connection section 10 includes marked lines 11 on both sides on outer surface and each line 11 has a plurality of equally spaced markings 13 along its length.

Preferably, the vein connection section 20 includes markings 21 at first and second ends 24, 25 respectively, two opposite lines 22 on an outer surface, each line 22 interconnecting the markings 21, and a plurality of equally spaced markings 23 along the line 22.

Preferably, the line 11 of the artery connection section 10 and the corresponding line 22 of the vein connection section 20 are joined at the enlargement 50. Preferably, the dialysis graft is made by Polytetraflouroethylene (PTFE).

Preferably, a distance between one ring 21 and the adjacent first end 24 of the vein connection section 20 is 0.5 cm, and a distance between the other ring 21 and the adjacent second end 25 of the vein connection section 20 is 0.5 cm. A distance between any two adjacent markings 13 or 23 is 0.5 cm. A distance between the ring 21 and the marking 23 adjacent thereto is 0.5 cm.

Preferably, the lines 11, 22, the marking 21, and the markings 13, 23 are marked with materials having color so that operator can see it during operation. In addition, these materials are not radiolucent so that they can be seen under fluoroscopy and X-rays. This can make that graft visible and lead easier for further endovascular therapy, such as percutaneous angioplasty (PTA).

Preferably, the artery connection section 10 is longer than the vein connection section 20.

Preferably, the artery connection section 10 and the vein connection section 20 are integral.

Preferably, the dialysis graft is T-shaped.

Preferably, the artery connection section 10 is inclined at an acute angle with respect to the vein connection section 20 at the connection.

Preferably, the enlargement 50 is formed at the obtuse angle side of the connection.

Preferably, an enlargement 50 is formed at the obtuse angle side of the connection.

Preferably, each line 22 of the vein connection section 20 is perpendicular to each marking 21.

The invention has the following advantages. The novel design of a dialysis graft can prevent thrombi formation, facilitate the blood flow through the circulatory system and reduce the cost of repeat thrombectomy. Moreover, a diameter of either end of the vein connection section 20 is equal to that of the corresponding section of the vein 40. That is, the connection of the vein connection section 20 and either section of the vein 40 has a smooth inner surface.

Marking lines 11, 22 provide orientation reference of graft that prevents the dialysis graft from twist during implantation and prevent graft caliber has been compromised. Further, the marking 21 can visually show a precise confluence site between the dialysis graft and the blood vessel. Furthermore, physicians can measure length of the dialysis graft inserted into the vein connection section 20 by counting the markings 13, 23 when a dialysis is being taken. This has the benefits of decreasing the difficulties of operation, decreasing operation time, and increasing the success probability of operation.

While the invention herein disclosed has been described by means of specific embodiments, numerous modifications and variations could be made thereto by those skilled in the art without departing from the scope and spirit of the invention set forth in the claims.

What is claimed is:

1. A dialysis graft comprising:
a vein connection section having first and second ends and including a smooth outer surface adapted to slip and connect to two sections of a vein respectively;
an artery connection section extending out of an intermediate portion of the vein connection section and communicating therewith, the artery connection section and the vein connection section forming a dialysis graft of T-shape, the artery connection section having a length greater than that of the vein connection section, the artery connection section having a third end connected to the artery, and the artery connection section being inclined at an angle with respect to the vein connection section at the connection to an artery to define an acute angle side and an obtuse angle side; and
an enlargement for preventing thromboses formed at the obtuse angle side of the connection;
wherein each of the artery and vein connection sections include marked lines on both sides on outer surface respectively, and each line has a plurality of equally spaced markings along its length;
wherein each of the lines of the artery connection section and the corresponding one of the lines of the vein connection section are joined at the enlargement;
wherein each of the artery and vein connection sections have a smooth inner surface;
wherein the vein connection section includes two ring-markings proximate both the first and second ends respectively; and
wherein each of the lines of the vein connection section is perpendicular to each of the ring markings.

2. The dialysis graft of claim 1, wherein the dialysis graft is made by Polytetraflouroethylene (PTFE).

3. The dialysis graft of claim 1, wherein a distance between the ring markings and the adjacent first or second end of the vein connection section is 0.5 cm.

4. The dialysis graft of claim 1, wherein a distance between any two adjacent ones of the markings is 0.5 cm.

5. The dialysis graft of claim 1, wherein a distance between the ring and the marking adjacent thereto is 0.5 cm.

6. The dialysis graft of claim 1, wherein the lines, the rings, and the markings are formed of materials visible in naked eyes and X-ray.
